(19) United States
(12) Patent Application Publication
(10) Pub. No.: US 2022/0110536 A1
(43) Pub. Date: Apr. 14, 2022

(54) METHODS AND SYSTEMS FOR MULTI-DIMENSIONAL PHOTOPLETHYSMOGRAPHY (PPG)

(71) Applicant: Bionous, LLC, Sammamish, WA (US)

(72) Inventor: Ali Bahrami, Sammamish, WA (US)

(73) Assignee: Bionous, LLC, Sammamish, WA (US)

(21) Appl. No.: 17/418,488

(22) PCT Filed: Dec. 27, 2019
(86) PCT No.: PCT/US2019/068694

§ 371 Date: Jun. 25, 2021

Related U.S. Application Data
(60) Provisional application No. 62/785,744, filed on Dec. 28, 2018.

(51) Int. Cl. A61B 5/024; A61B 5/0205; A61B 5/021; A61B 5/145; A61B 5/1455; A61B 5/08; A61B 5/00

(52) U.S. Cl. CPC A61B 5/02433; A61B 5/02055; A61B 5/021; A61B 5/14546; A61B 5/6826; A61B 5/1455; A61B 5/0816; A61B 5/7235; A61B 2562/166; A61B 5/14532

(57) ABSTRACT

Methods and apparatus for acquiring a noninvasive multi-dimensional photoplethysmogram (PPG) are provided. The apparatus comprises a plurality of light emitting diodes (LEDs), wherein a first LED of the plurality of LEDs is a first member of the group consisting of a green LED, a red LED, and an infrared LED and a second LED of the plurality of LEDs is a second member of a group consisting of a green LED, a red LED, and an infrared LED, wherein the plurality of LEDs are configured to illuminate a tissue, at least one sensor configured to sense light reflected from the tissue in response to illumination of the tissue by the plurality of LEDs, and at least one processor programmed to determine a multi-dimensional photoplethysmogram based, at least in part, on the sensed reflected light.

### CROSS-REFERENCE TO RELATED APPLICATIONS

This application claims the benefit under 35 U.S.C. § 119(e) of U.S. Provisional Application No. 62/785,744, entitled “METHODS AND SYSTEMS OF 3D PHOTOPLETHYSMOGRAM (PPG),” filed Dec. 28, 2018, the entire contents of which is incorporated by reference herein.

### BACKGROUND

Photoplethysmography (PPG) is a noninvasive diagnostic technique for estimating changes in blood volume in the microvascular bed of tissue (e.g., skin tissue). In PPG, tissue is illuminated with light and the reflected light is measured. A photoplethysmogram may be obtained using a pulse oximeter, which illuminates the skin and measures changes in light absorption to determine oxygen saturation and heart rate.

### SUMMARY

Some embodiments relate to an apparatus configured to acquire a noninvasive multi-dimensional photoplethysmogram (PPG). The apparatus comprises a plurality of light emitting diodes (LEDs), wherein a first LED of the plurality of LEDs is a first member of the group consisting of a green LED, a red LED, and an infrared LED and a second LED of the plurality of LEDs is a second member of a group consisting of a green LED, a red LED, and an infrared LED, wherein the plurality of LEDs are configured to illuminate a tissue, at least one sensor configured to sense light reflected from the tissue in response to illumination of the tissue by the plurality of LEDs, and at least one processor programmed to determine a multi-dimensional photoplethysmogram based, at least in part, on the sensed reflected light.

In one aspect, the plurality of LEDs includes at least one green LED, at least one red LED, and at least one infrared LED, and wherein the multi-dimensional photoplethysmogram is a three-dimensional photoplethysmogram.

In another aspect, the at least one processor is further programmed to determine one or more biological quantities based on the multiple-dimensional photoplethysmogram.

In another aspect, the one or more biological quantities includes blood pressure, blood sugar, respiration information, body temperature information, and/or cholesterol information.

In another aspect, the at least one processor is further programmed to determine at least one biological characteristic based, at least in part on a change of patterns observed in variations of the sensed reflected light.

In another aspect, the at least one processor is further programmed to vectorize the multi-dimensional photoplethysmogram, and determine at least one biological quantity based on the vectorized multi-dimensional photoplethysmogram.

Some embodiments relate to a method of noninvasively acquiring a multi-dimensional photoplethysmogram (PPG). The method comprises illuminating a tissue with a plurality of light emitting diodes (LEDs), wherein a first LED of the plurality of LEDs is a first member of the group consisting of a green LED, a red LED, and an infrared LED and a second LED of the plurality of LEDs is a second member of a group consisting of a green LED, a red LED, and an infrared LED, sensing, using at least one sensor, light reflected from the tissue in response to illumination of the tissue by the plurality of LEDs, and determining, using at least one processor, a multi-dimensional photoplethysmogram based, at least in part, on the sensed reflected light.

In one aspect, the plurality of LEDs includes at least one green LED, at least one red LED, and at least one infrared LED, and wherein determining the multi-dimensional photoplethysmogram comprises determining a three-dimensional photoplethysmogram.

In another aspect, the method further comprises determining one or more biological quantities based on the multiple-dimensional photoplethysmogram.

In another aspect, the one or more biological quantities includes at least one vital sign selected from the group consisting of blood pressure, blood sugar, respiration information, body temperature information, and cholesterol information.

In another aspect, the method further comprises determining at least one biological characteristic based, at least in part on a change of patterns observed in variations of the sensed reflected light.

In another aspect, the method further comprises vectorizing the multi-dimensional photoplethysmogram, and determining at least one biological quantity based on the vectorized multi-dimensional photoplethysmogram.

Some embodiments relate to at least one non-transitory computer readable medium having encoded thereon, a plurality of instructions that, when executed by at least one processor, perform a method of noninvasively acquiring a multi-dimensional photoplethysmogram (PPG). The method comprises illuminating a tissue with a plurality of light emitting diodes (LEDs), wherein a first LED of the plurality of LEDs is a first member of the group consisting of a green LED, a red LED, and an infrared LED and a second LED of the plurality of LEDs is a second member of a group consisting of a green LED, a red LED, and an infrared LED, sensing, using at least one sensor, light reflected from the tissue in response to illumination of the tissue by the plurality of LEDs, and determining, using at least one processor, a multi-dimensional photoplethysmogram based, at least in part, on the sensed reflected light.

In one aspect, the plurality of LEDs includes at least one green LED, at least one red LED, and at least one infrared LED, and wherein determining the multi-dimensional photoplethysmogram comprises determining a three-dimensional photoplethysmogram.

In another aspect, the method further comprises determining one or more biological quantities based on the multiple-dimensional photoplethysmogram.

In another aspect, the one or more biological quantities includes at least one vital sign selected from the group consisting of blood pressure, blood sugar, respiration information, body temperature information, and cholesterol information.

In another aspect, the method further comprises determining at least one biological characteristic based, at least in part on a change of patterns observed in variations of the sensed reflected light.

In another aspect, the method further comprises vectorizing the multi-dimensional photoplethysmogram, and determining at least one biological quantity based on the vectorized multi-dimensional photoplethysmogram.

Some embodiments relate to printed circuit board having integrated thereon: a plurality of light emitting diodes (LEDs), wherein a first LED of the plurality of LEDs is a first member of the group consisting of a green LED, a red LED, and an infrared LED and a second LED of the plurality of LEDs is a second member of a group consisting of a green LED, a red LED, and an infrared LED, wherein the plurality of LEDs are configured to illuminate a tissue, and at least one processor programmed to determine a multi-dimensional photoplethysmogram based, at least in part, on light reflected from the tissue in response to illumination of the tissue by the plurality of LEDs.

Some embodiments relate to an apparatus configured to acquire a noninvasive 3D photoplethysmogram (PPG), the apparatus comprising: green, red, and infrared light emitting diodes configured to illuminate a tissue; at least one sensor configured to sense reflected green, red, and infrared light from the sample; and at least one processor programmed to determine a 3D photoplethysmogram based, at least in part, on the sensed green, red, and infrared light.

In one aspect, the at least one processor is further programmed to determine blood pressure based, at least in part, on the 3D photoplethysmogram.

In another aspect, the at least one processor is further programmed to determine blood sugar based, at least in part, on the 3D photoplethysmogram.

In another aspect, the at least one processor is further programmed to determine respiration information based, at least in part, on the 3D photoplethysmogram.

In another aspect, the at least one processor is further programmed to determine body temperature information based, at least in part, on the 3D photoplethysmogram.

In another aspect, the at least one processor is further programmed to determine cholesterol information based, at least in part, on the 3D photoplethysmogram.

In another aspect, the at least one processor is further programmed to determine at least one biological characteristic based, at least in part on a change of patterns observed in variations of the reflected green, red and infrared light.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

### BRIEF DESCRIPTION OF DRAWINGS

Various non-limiting embodiments of the technology will be described with reference to the following figures. It should be appreciated that the figures are not necessarily drawn to scale.

FIG. 1 is a plot of a photoplethysmogram (PPG) signal recorded using a single LED PPG system;

FIGS. 2A and 2B illustrate, respectively, a transmission-based, and a reflective-based PPG system;

FIG. 3 schematically illustrates a spectrum of visible light, where different wavelengths of light is associated with a particular color;

FIG. 4 is a plot showing different optical skin penetration depths for different light sources;

FIGS. 5A and 5B show respectively, a photograph and a schematic illustration of PPG circuit that includes multiple LED sources in accordance with some embodiments;

FIG. 6 is a plot of a multi-dimensional PPG measurement in accordance with some embodiments;

FIG. 7 is a plot showing some characteristics of one cycle of a PPG measurement that can be used to determine one or more biological quantities in accordance with some embodiments;

FIG. 8 is a plot showing additional characteristics of a PPG measurement that can be used to determine one or more biological quantities in accordance with some embodiments;

FIG. 9 is a visualization of a 3D PPG measurement in accordance with some embodiments;

FIG. 10 is a plot of a vectorization of features of a multi-dimensional PPG in accordance with some embodiments; and

FIG. 11 is an illustrative process for determining at least one biological quantity from a multi-dimensional PPG in accordance with some embodiments.

### DETAILED DESCRIPTION

A conventional pulse oximeter used to perform PPG measurement monitors the perfusion of blood to the dermis and subcutaneous tissue of the skin. With each cardiac cycle, the heart pumps blood to the periphery. Even though this pressure pulse may be damped by the time it reaches the skin, the pressure pulse is strong enough to distend the arteries and arterioles in the subcutaneous tissue. If the pulse oximeter is attached without compressing the skin, a pressure pulse can also be observed from the venous plexus, as a small secondary peak in the signal.

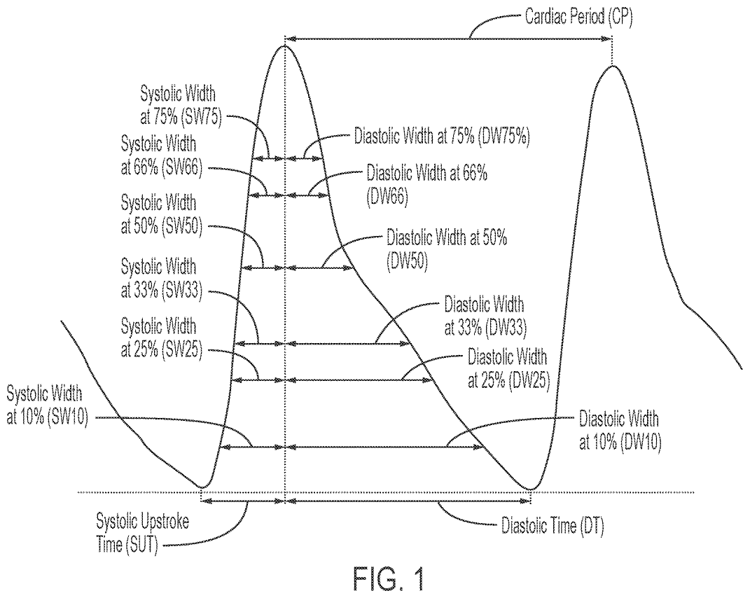

The change in volume caused by the pressure pulse is detected by illuminating the skin with light from a light source (e.g., a light-emitting diode (LED)), and measuring the amount of light either transmitted or reflected to a photosensitive device (e.g., a photodiode). Each cardiac cycle appears as a peak in the signal output from the photodiode, as shown in FIG. 1. Because blood flow to the skin may be modulated by multiple other physiological systems, the PPG may also be used to, in addition to cardiac monitoring, monitor breathing, hypovolemia, and other circulatory conditions. Additionally, the shape of the PPG waveform differs from subject to subject, and varies with the location and manner in which the pulse oximeter is attached.

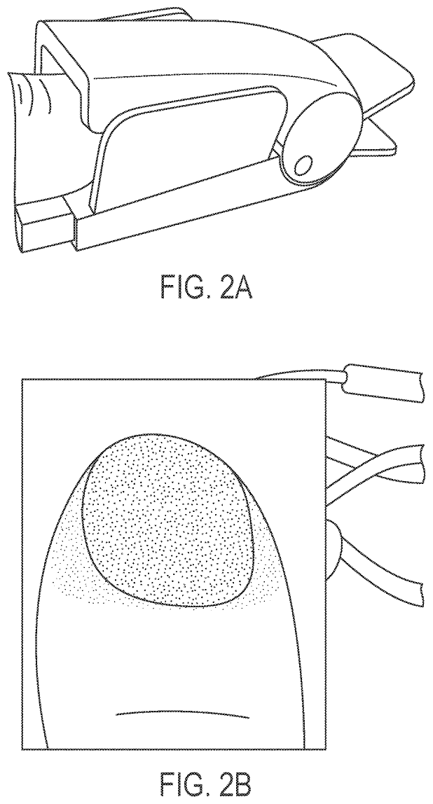

A typical optical system for peripheral capillary oxygen saturation (SpO₂) measurement includes light emitting diodes (LEDs) that illuminate tissue and a photodiode that receives the transmitted or reflected light. Two types of optical arrangements for performing SpO₂ measurements are transmissive and reflective. For transmissive arrangements, the photodiode and the LED are placed on opposite sides of a human body part (e.g., a finger), with the photodiode collecting the residual light after absorption from the various components of the body part. For reflective arrangements, the photodiode and the LED are placed on the same side of the illuminated body part and the photodiode collects the light reflected from various depths underneath the skin. Finger-clip type probes commonly used in a medical clinic, an example of which is shown in FIG. 2A, are typical of the transmissive type. In such probes, photons in the light output from the LED scatter in every direction when they encounter an object, for example, blood cells in a person's finger. Although the separation between the LED source and the photodiode is typically around 10 mm, most of the photons output from the LED source travel 10-20 cm before reaching the photodiode due to scattering within the illuminated tissue, with some photons potentially travelling as far as 200 mm. The path of the photons scattering within the tissue may be described as walking randomly, which is often observed as a characteristic glow in the skin of the fingertip, for example, as shown in FIG. 2B.

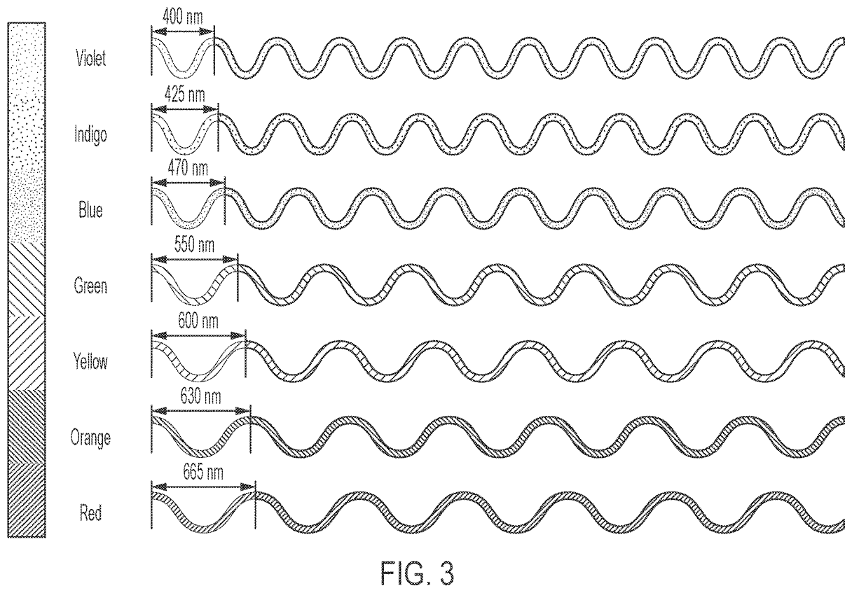

FIG. 3 is a schematic illustration which shows that light is composed of wavelengths of light, and each wavelength is associated with a particular color. The observable color of an object depends on which wavelengths of the light are reflected from the object to the eyes of the observer.

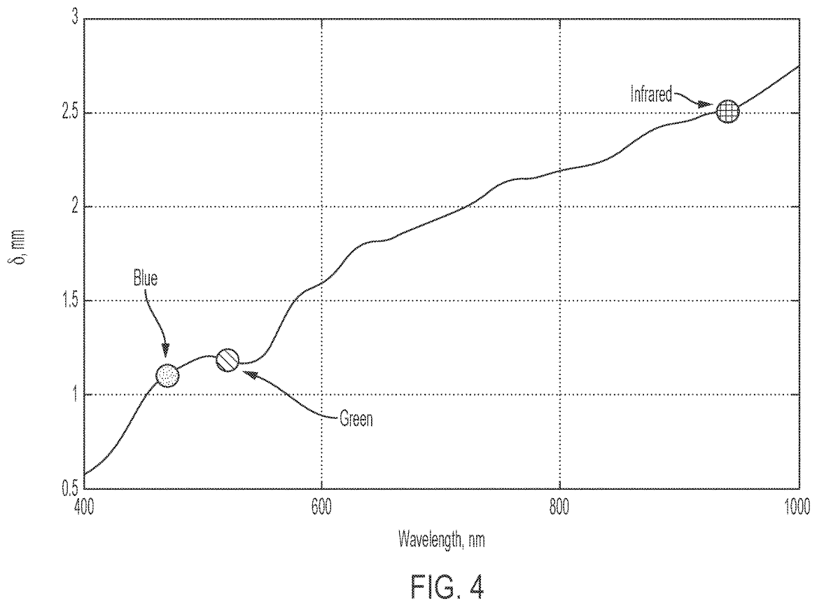

For PPG measurements, the penetration depth of light into tissues depends, at least in part, on the wavelength of the light used for illuminating the tissue. FIG. 4 shows the skin penetration depth δ for light wavelengths ranging from 400 to 1000 nm. For instance, light characterized by shorter wavelengths (e.g., green light) penetrates soft tissue less than light characterized by longer wavelengths (e.g., infrared).

Infrared and red-light PPG sensors (also commonly-referred to as “pulse oximeters”) typically utilize light in the near-infrared spectroscopy (NIRS) range (e.g., from 780-2500 nm) and are widely used in medical offices and hospitals, where accuracy is closely monitored. The human body does not absorb light in the NIRS range well and allows the transmission of such light to penetrate 10× deeper into multiple tissue layers in comparison to shorter wavelength (e.g., green) light. Additionally, tattoos, freckles, and melanin in the skin do not typically affect measurements by red light sensors.

Some reflective-type arrangements for PPG measurements have used a green LED source (i.e., an LED source configured to produce light in the green wavelength range of approximately 550 nm) to extract the PPG signal from soft tissue. Due to its shorter wavelength, green light penetrates the tissues less than LEDs configured to output longer-wavelength light such as red or infrared LEDs. Hence, more unabsorbed (reflected) light is observed when green LEDs are used as the light source compared to when light sources with longer wavelengths (e.g., red or infrared LEDs) are used.

Green light PPG sensors are sometimes used in optical heart rate monitor (OHRM) products. Reasons for using green light PPG sensors as opposed to red or infrared-light PPG sensors include a greater signal-to-noise ratio and more resistance to motion artifacts. For example, because skin absorbs green light from the LEDs very well, using green light LEDs limits the amount of light that passes through the tissue thereby improving the signal-to-noise ratio of the overall reflected light.

Although there are some benefits to using green light for PPG measurements, there are also some drawbacks. For instance, skin tone, specifically the amount of melanin, affects the skin's ability to absorb green light and further increases the variation in reporting accuracy. Additionally, hemoglobin strongly absorbs green light, resulting in green light being unable to reach deeper tissues to extract insights about those deeper tissues.

The inventor has recognized and appreciated that some conventional PPG systems may be improved by providing a system that includes multiple different types of LED sensors, which provide complimentary information. In particular, some embodiments relate to using multi-dimensional (e.g., three-dimensional (3D)) photoplethysmogram (PPG), in which PPG measurement are made at different depths within the tissue being interrogated. In testing using 3D PPG, the accuracy of the prediction model for measuring vital signs such as blood pressure, blood sugar, cholesterol was increased by 15 to 20 percent compared to conventional PPG techniques (e.g., using only a single type of LED).

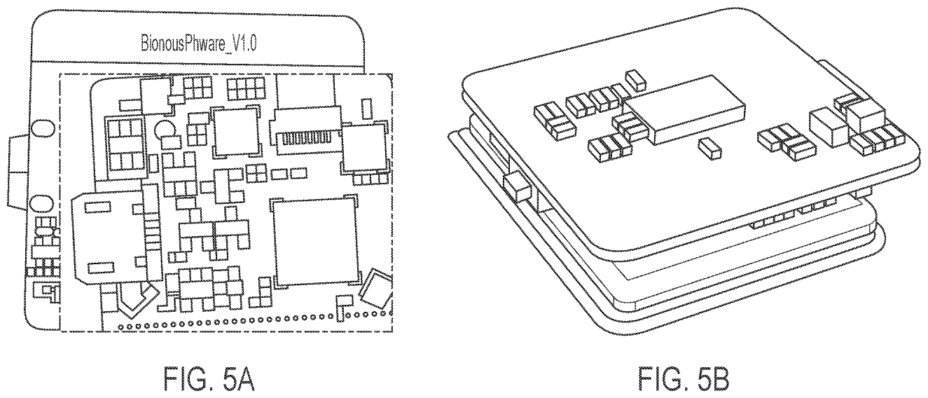

FIGS. 5A and 5B show, respectively, a photograph and a schematic of example circuitry for a multi-dimensional PPG system in accordance with some embodiments. Such circuitry may include multiple types of LEDs integrated thereon. For instance, in some embodiments, the circuitry includes green, red and infrared LEDs. Other circuitry components including, but not limited to, sensors and an embedded processor (e.g., a central processing unit (CPU)) for processing signals output from the sensors may also be integrated with the circuitry in addition to the different types of LEDs.

In some embodiments, the multi-dimensional PPG system includes at least one green LED configured to output light having a wavelength of approximately 550 nm, at least one red LED configured to output light having a wavelength of approximately 660 nm and at least one infrared LED configured to output light having a wavelength of approximately 950 nm. The inclusion of multiple types of LEDs configured to produce light having different wavelengths provides the multi-dimensional PPG system with the ability to collect information from different depths within the soft tissue due to the different penetration depths of the respective LEDs. In embodiments that include at least one green LED, at least one red LED, and at least one infrared LED, the variation of the depth of penetration from the different types of LED sources provides three points of view to observe, collect and process PPG features, resulting in a higher accuracy in estimating PPG features compared to a PPG device with a single type/color of LED.

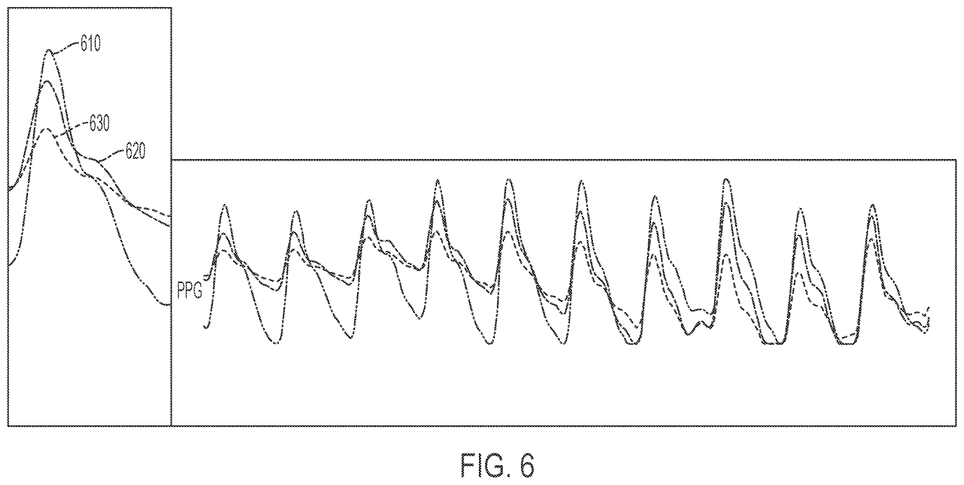

FIG. 6 shows an example of multi-dimensional PPG signals recorded in accordance with some embodiments. As shown, signals corresponding to each of the different types of LED sources have peaks that roughly correspond in time, but have different amplitudes and shapes that provide complimentary information about the soft tissue being interrogated with the PPG system. For instance, the green signal 610 corresponding to reflected green light has a larger amplitude compared to the infrared signal 620 corresponding to reflected infrared light or the red signal 630 corresponding to reflected red light.

Some embodiments include dedicated hardware, firmware and/or software for processing the PPG signals to determine one or more biological quantities or characteristics, examples of which are described in more detail below.

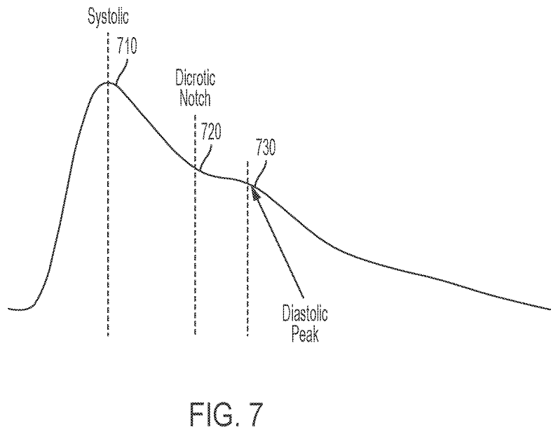

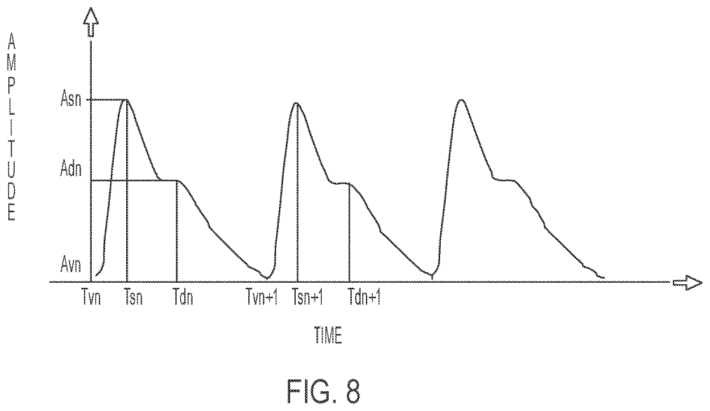

As shown in FIG. 7, a cycle of the PPG signal includes at least three features—the systolic peak 710, the dicrotic notch 720, and the diastolic peak 730. As shown in FIG. 8, these features repeat every cycle of the PPG signal and further parameters (e.g., related to timing and amplitude of the signal) can be determined including systolic peak parameters (Tsn, Asn), valley point parameters (Tvn, Avn) and dicrotic notch parameters (Tdn, Adn). These point features may further be used to compute PPG derivative features.

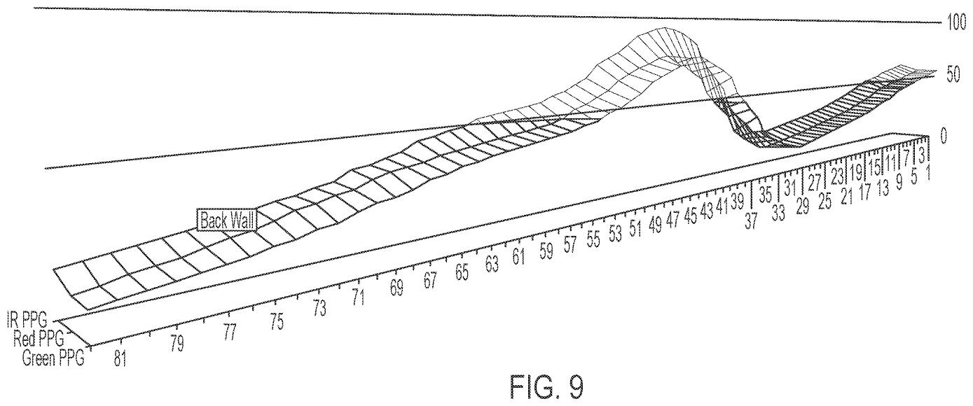

In some embodiments, rather than having a single point feature at every time t, a multi-dimensional PPG, an example of which is shown in FIG. 9, includes a vector with multiple (e.g., three) dimensions at each time t. These vectors (e.g., a green PPG, a red PPG, and an infrared PPG) provide enriched data compared to single point PPG. In some embodiments, the vectors in the multi-dimensional PPG are further processed, for example, using a machine learning model or another PPG processing algorithm. In some embodiments, the further processing of the PPG vectors is performed by at least one embedded processor integrated on a same circuit substrate as the LED sources.

In some instances, variations between the PPG signals (e.g., for green, red, and infrared LEDs) are dynamic where differences can be visually observed. In other instances, the variations between the PPG signals may not be visually observable. In some embodiments, the multi-dimensional PPG system may be transformed into a higher dimensional PPG system (e.g., from a 3D system to a 4D system) in which the change of patterns across the multiple PPG signals provides the fourth dimension. This variation creates depth in PPG core feature computations and can also improve on accuracy of the PPG based algorithms.

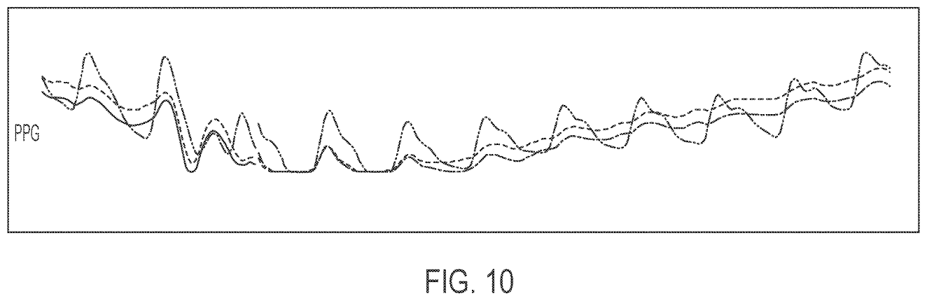

In mathematics, the vectorization of a matrix is a linear transformation which converts the matrix into a column vector. Here the concept of vectorization has been applied to transforming one or more of PPG's features such as Avn, Adn, Asn, Tvn, Tsn and Tvn. These core features can be transformed to vectors for instance Avn to vec(Avn), Adn to vec(Adn), Asn to vec(Asn), Tvn to vec(Tvn), Tsn to vec(Tsn), and Tvn to vec(Tvn). FIG. 10 illustrates vectorization of PPG features for the three PPG signals shown in FIG. 6 in accordance with some embodiments.

Once PPG features have been vectorized, any mathematical operations from add, subtract, multiply, average or user define operations can be performed on the vectorized PPG features.

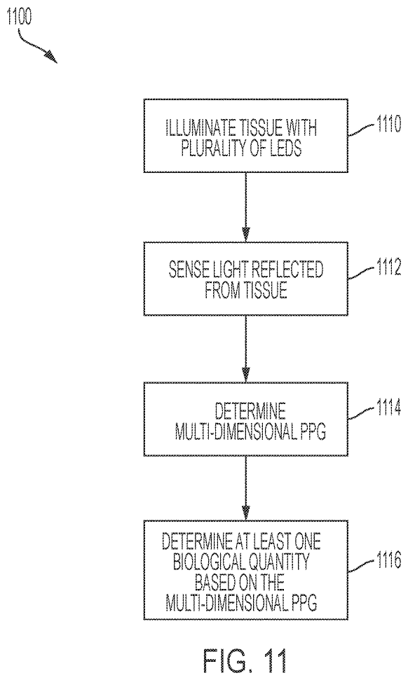

FIG. 11 shows an illustrative process 1100 for determining one or more biological quantities based on acquiring a multi-dimensional photoplethysmogram (PPG) in accordance with some embodiments. In act 1110, a tissue (e.g., soft tissue of a human or other animal) is illuminated with light from a plurality of LEDs. The plurality of LEDs include at least two types of LEDs configured to output light having different wavelengths. For instance, the plurality of LEDs may include a green LED, a red LED, and an infrared LED or any combination of these LEDs. As described above, including measurements from LEDs configured to output light having different wavelengths permits probing the tissue at different tissue depths, which provides more and complimentary information than is possible when only a single type of LED is used.

Process 1100 then proceeds to act 1112, where light reflected from the tissue in response to the illumination by the plurality of LEDs is sensed. One or more sensors may be used to detect the reflected light and embodiments are not limited in this respect. For instance, in some embodiments, a single sensor that includes filters for filtering the reflected light may be used. In other embodiments, multiple sensors, each of which is configured to recognize a single type of reflected light may be used.

Process 1100 then proceeds to act 1114, where a multi-dimensional (e.g., 3-dimensional or 3D) PPG is determined based, at least in part, on the sensed reflected light. Examples of illustrative multi-dimensional PPG are shown in connection with FIGS. 6 and 9 discussed above.

Process 1100 then proceeds to act 1116, where one or more biological quantities or characteristics are determined based, at least in part, on the multi-dimensional PPG determined in act 1114. Any suitable biological quantities or characteristics may be determined, non-limiting examples of which include blood pressure, blood sugar, respiration information, body temperature information, and cholesterol information. In this way, noninvasive PPG measurements having a high accuracy may be obtained.

The above-described embodiments can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware or with one or more processors programmed using microcode or software to perform the functions recited above.

In this respect, it should be appreciated that one implementation of the embodiments of the present invention comprises at least one non-transitory computer-readable storage medium (e.g., a computer memory, a portable memory, a compact disk, a tape, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

1.-23. (canceled)

24. An apparatus configured to non-invasively acquire a multi-dimensional photoplethysmogram (PPG), the apparatus comprising:
a plurality of light emitting diodes (LEDs) configured to illuminate a tissue, wherein the plurality of LEDs include a green LED, a red LED, and an infrared LED;
at least one sensor configured to sense light reflected from the tissue as a plurality of signals, wherein each of the plurality of signals corresponds to reflected light sensed in response to illumination of the tissue by one of the green LED, the red LED or the infrared LED; and
at least one processor programmed to:
combine the plurality of signals into a multi-dimensional photoplethysmogram (PPG) wherein each of a plurality of timepoints of the multi-dimensional PPG includes a plurality of values, each of the plurality of values corresponding to one of the plurality of signals;
vectorize the multi-dimensional PPG and/or one or more features extracted from the multi-dimensional PPG; and
determine one or more biological quantities based on the vectorized multi-dimensional PPG and/or the vectorized one or more features extracted from the multi-dimensional PPG.

25. The apparatus of claim 24, wherein the one or more biological quantities includes blood pressure, blood sugar, respiration information, body temperature information, and cholesterol information.

26. The apparatus of claim 24, wherein the at least one processor is further programmed to:
determine a change of patterns across the plurality of signals included in the multi-dimensional PPG;
transform the multi-dimensional PPG into a higher-dimensional PPG system; and
determine the one or more biological quantities based, at least in part on the higher-dimensional PPG system.

27. The apparatus of claim 24, wherein the plurality of LEDs, the at least one sensor and the at least one processor are integrated on a printed circuit board.

28. A method of noninvasively acquiring a multi-dimensional photoplethysmogram (PPG), the method comprising:
illuminating a tissue with a plurality of light emitting diodes (LEDs), the plurality of LEDs including a green LED, a red LED, and an infrared LED;
sensing, using at least one sensor, light reflected from the tissue as a plurality of signals, wherein each of the plurality of signals corresponds to reflected light sensed in response to illumination of the tissue by one of the green LED, the red LED or the infrared LED;
combining, using at least one processor, the plurality of signals into a multi-dimensional photoplethysmogram (PPG), wherein each of a plurality of timepoints of the multi-dimensional PPG includes a plurality of values, each of the plurality of values corresponding to one of the plurality of signals;
vectorizing the multi-dimensional PPG and/or one or more features extracted from the multi-dimensional PPG; and
determining one or more biological quantities based on the vectorized multi-dimensional PPG and/or the vectorized one or more features extracted from the multi-dimensional PPG.

29. The method of claim 28, wherein the one or more biological quantities includes blood pressure, blood sugar, respiration information, body temperature information, and cholesterol information.

30. The method of claim 28, further comprising:
determining a change of patterns across the plurality of signals included in the multi-dimensional PPG;
transforming the multi-dimensional PPG into a higher-dimensional PPG system; and
determining the one or more biological quantities based, at least in part on the higher-dimensional PPG system

31. At least one non-transitory computer readable medium having encoded thereon, a plurality of instructions that, when executed by at least one processor, perform a method of noninvasively acquiring a multi-dimensional photoplethysmogram (PPG), the method comprising:
illuminating a tissue with a plurality of light emitting diodes (LEDs), the plurality of LEDs including a green LED, a red LED, and an infrared LED;
sensing, using at least one sensor, light reflected from the tissue as a plurality of signals, wherein each of the plurality of signals corresponds to reflected light sensed in response to illumination of the tissue by one of the green LED, the red LED or the infrared LED;
combining, using at least one processor, the plurality of signals into a multi-dimensional photoplethysmogram (PPG), wherein each of a plurality of timepoints of the multi-dimensional PPG includes a plurality of values, each of the plurality of values corresponding to one of the plurality of signals;
vectorizing the multi-dimensional PPG and/or one or more features extracted from the multi-dimensional PPG; and
determining one or more biological quantities based on the vectorized multi-dimensional PPG and/or the vectorized one or more features extracted from the multi-dimensional PPG.

32. The at least one non-transitory computer readable medium of claim 31, wherein the one or more biological quantities includes blood pressure, blood sugar, respiration information, body temperature information, and cholesterol information.

33. The at least one non-transitory computer readable medium of claim 31, wherein the method further comprises:
determining a change of patterns across the plurality of signals included in the multi-dimensional PPG;
transforming the multi-dimensional PPG into a higher-dimensional PPG system; and
determining the one or more biological quantities based, at least in part on the higher-dimensional PPG system
